(19) United States
(12) Patent Application Publication
(10) Pub. No.: US 2021/0005296 A1
(43) Pub. Date: Jan. 7, 2021

(54) SYSTEM AND METHOD FOR DETERMINING BEST PRACTICES FOR THIRD PARTIES ACCESSING A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 16/656,220

(22) Filed: Oct. 17, 2019

Related U.S. Application Data
(60) Provisional application No. 62/736,351, filed on Sep. 25, 2018.

(51) Int. Cl. G16H 10/60; G06Q 20/22; H04W 12/04

(52) U.S. Cl. CPC G16H 10/60; H04W 12/04031; G06Q 20/22

(57) ABSTRACT

A system and a method for determining best practices for third parties accessing a health care network are described. The method comprises providing a user device for allowing a patient to connect to a health care network implemented over blockchain, providing a third party device for allowing the third parties to connect to the health care network. The method further comprises receiving, from a third party, a request for accessing data of the patient and a first parameter related to the patient. The first parameter is correlated with patients' data, using an Artificial Intelligence (AI) engine, to identify a second parameter relevant for the patient. The second parameter is provided to the third party, for being used as a best practice by the third party.

### OTHER RELATED APPLICATIONS

The present application is a U.S. Non-Provisional Patent Application claiming priority of U.S. Provisional Patent Application Ser. No. 62/736,351 filed on Sep. 25, 2018, which is hereby incorporated by reference.

### BACKGROUND OF THE INVENTION

### 1. Field of the Invention

The present disclosure is generally related to data processing in a healthcare network implemented over blockchain, and more particularly related to processing of the data using Artificial Intelligence for determining best practices.

### 2. Description of the Related Art

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner. The blocks are added to the blockchain in a linear and chronological order. As the entirety of sensitive information present on the blockchain (data) is present over several blocks, it is difficult to gather the data at a single place and thereupon analyze or process such data for meeting specific requirements while also managing data access by interested users. Thus, there exists a need for more effectively and efficiently managing access of the data and processing the data to facilitate user's requirements.

Applicant believes that a related reference corresponds to U.S. Pat. No. 10,340,038 for Healthcare Transaction Validation via Blockchain, Systems and Methods. Applicant believes another related reference corresponds to U.S. patent No. 2007/0061393 for Management of Health Care Data. None of these references, however, teach of being able to call upon data in a quick and easy manner in a blockchain even when the data for one particular patient has not been stored and input all on one same day. Further, the patient data as it is sensitive information is encrypted, yet the delay for obtaining the required data is minimal.

Other documents describing the closest subject matter provide for a number of more or less complicated features that fail to solve the problem in an efficient and economical way. None of these patents suggest the novel features of the present invention.

### SUMMARY OF THE INVENTION

It is one of the objects of the present invention to provide a system and method for determining best practices for third parties accessing a health care network that can collect private and sensitive data.

It is another object of this invention to provide a system and method for determining best practices for third parties accessing a health care network that can securely store collected data by means of encryption.

It is still another object of the present invention to provide a system and method for determining best practices for third parties accessing a health care network that allows access to stored data remotely by appropriate parties, such as patients, health care providers, physicians and the like.

It is another object of the present invention to provide a system and method for determining best practices for third parties accessing a health care network capable of using artificial intelligence.

It is still another object of the present invention to provide a system and method for determining best practices for third parties accessing a health care network that uses artificial intelligence to analyze patient data collected.

It is still another object of the present invention to provide a system and method for determining best practices for third parties accessing a health care network that uses artificial intelligence to correlate patient data to predetermined parameters.

It is yet another object of the present invention to provide a system and method for determining best practices for third parties accessing a health care network that collects data in a blockchain in a linear and chronological order yet allows for easily accessing of collected data spanning some time period at a particular place and time.

It still another object of the present invention to provide a system and method for determining best practices for third parties accessing a health care network that can be accessed remotely.

It is another object of the present invention to provide a system and method for determining best practices for third parties accessing a health care network that allows for effectively and efficiently managing accessing of collected data.

It is yet another object of this invention to provide a method and system that is inexpensive to implement and maintain while retaining its effectiveness.

Further objects of the invention will be brought out in the following part of the specification, wherein detailed description is for the purpose of fully disclosing the invention without placing limitations thereon.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram of a Health Information Exchange (HIE) system for determining third party access to a health care network, according to various embodiments.

FIG. 2 illustrates a method for symmetric encryption of data, according to various embodiments.

FIG. 2A illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in a health care network implemented over a blockchain network, according to various embodiments.

FIG. 6 illustrates a flowchart showing an example process carried out by a patient setup module, according to various embodiments.

FIG. 7 illustrates a flowchart showing an example process carried out by a machine learning module, according to various embodiments.

FIG. 8 illustrates exemplary data stored in an artificial intelligence learning module (AILM) patient database, according to various embodiments.

FIG. 9 illustrates a flowchart showing an example process carried out by a machine learning algorithm module, according to various embodiments.

FIG. 10 illustrates exemplary data stored in a machine learning database.

FIG. 10A illustrates exemplary data stored in a machine learning database.

FIG. 11 illustrates a flowchart showing an example process carried out by a patient module, according to various embodiments.

### DETAILED DESCRIPTION OF THE EMBODIMENTS OF THE INVENTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of various embodiments of the present disclosure, various embodiments of the systems and methods will be described.

Embodiments of the present disclosure will be described more fully hereinafter with reference to the accompanying drawings in which like numerals may represent like elements throughout the several figures, and in which various example embodiments are shown. Various embodiments may, however, be embodied in many different forms and should not be construed as limited to the embodiments set forth herein. The examples set forth herein are non-limiting examples and are merely examples among other possible examples.

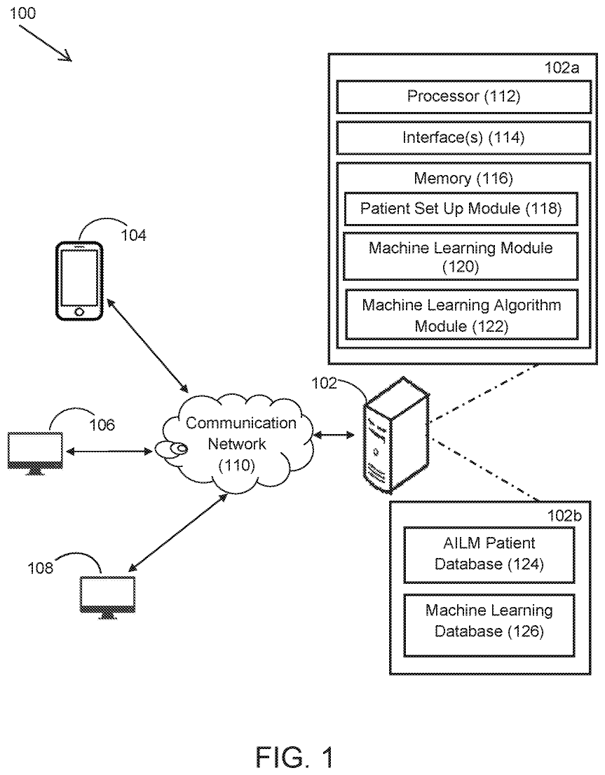

FIG. 1 illustrates a network connection diagram 100 of a Health Information Exchange (HIE) system 102 for determining third party access to a health care network, or best practices for a third parties accessing the health care network. HIE system 102 may comprise one or more user interfaces. The one or more user interfaces may be accessed by one or more users via one or more devices. The one or more device may comprise, for example, a user device 104, a doctor device 106, and a third-party device 108. HIE system 102 may be connected with user device 104, doctor device 106, and third-party device 108, through a communication network 110.

Communication network 110 may be a wired and/or a wireless network. Communication network 110, if wireless, may be implemented using communication techniques such as Visible Light

Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

HIE system 102 may comprise a group of components 102a for determining third party access to the health care network, or best practices for a third parties accessing the health care network. Group of components 102a may include a processor 112, interface(s) 114, and a memory 116. Memory 116 may comprise a patient set up module 118, a machine learning module 120, and a machine learning algorithm module 122. Further, HIE system 102 may include or may be connected with a group of databases 102b which may include an Artificial Intelligence Learning Module (AILM) patient database 124 and a machine learning database 126. In various embodiments, patient set up module 118, machine learning module 120, machine learning algorithm module 122, AILM database 124, and machine learning database 126 may be a part of an AI (Artificial Intelligence) module.

Processor 112 may execute an algorithm stored in memory 116 for determining best practices for third parties accessing a health care network. Processor 112 may also be configured to decode and execute any instructions received from one or more other electronic devices or server(s). Processor 112 may include one or more general purpose processors (e.g., microprocessors) and/or one or more special purpose processors (e.g., digital signal processors (DSPs), System On Chips (SOCs), Field Programmable Gate Arrays (FPGAs), or Application-Specific Integrated Circuits (ASICs)). Processor 112 may be configured to execute one or more computer-readable program instructions, such as program instructions to carry out any of the functions described in this description.

Interface(s) 114 may help an operator to interact with HIE system 102. Interface(s) 114 may either accept inputs from users or provide outputs to the users or may perform both the actions. In various embodiments, a user can interact with interface(s) 114 using one or more user-interactive objects and devices. The user-interactive objects and devices may comprise user input buttons, switches, knobs, levers, keys, trackballs, touchpads, cameras, microphones, motion sensors, heat sensors, inertial sensors, touch sensors, or any combination of the above. Further, interface(s) 114 may be implemented as a Command Line Interface (CLI), a Graphical User Interface (GUI), a voice interface, or a web-based user-interface.

Memory 116 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions. Memory 116 may comprise modules implemented as a program. As mentioned above, memory 116 may comprise patient setup module 118, machine learning module 120, and machine learning algorithm module 122.

In various embodiments, several users may interact with HIE system 102, using user device 104. Although a single user device 104 has been illustrated, several of user device 104 could similarly be connected to communication network 110. Further, each of user device 104 may have a device ID. In various embodiments, the device ID may be a unique identification code such as an International Mobile Equipment Identity (IMEI) code or a product serial number. It should be noted that a user may use a single of user device 104 or multiple of user device 104. Further, multiple users may use a single user device 104 or multiple of user device 104. Further, the one or more users may receive and/or provide healthcare related products and services. The one or more users may include, for example and not limited to, patients, family and friends of the patients, hospitals, physicians, nurses, specialists, pharmacies, medical laboratories, testing centers, insurance companies, or Emergency Medical Technician (EMT) services.

User device 104 may be a stationary device, a portable device, or a device accessed remotely. User device 104 may be, but not limited to, a computer, a laptop, a tablet, a mobile phone, a smartphone, or a smart watch. In various embodiments, user device 104 may include an imaging device that may be configured to capture a visual graphical element. The visual graphical element may be, for example but not limited to, a barcode, text, a picture, or any other forms of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to user device 104. In various embodiments, the hardware camera sensor may be embedded in user device 104. In various embodiments, the imaging device may be located external to user device 104. In various embodiments, the imaging device may be connected to user device 104 wirelessly or via a cable. It should be noted that image data of the visual graphical element may be transmitted to user device 104 via communication network 110.

In various embodiments, the imaging device may be controlled by applications and/or software(s) configured to scan a visual graphical code. In various embodiments, a camera may be configured to scan a QR code. Further, the applications and/or software(s) may be configured to activate the camera present in user device 104 to scan the QR code. In various embodiments, the camera may be controlled by a processor natively embedded in user device 104. In various embodiments, the imaging device may include a screen capturing software, for example, a screenshot that may be configured to capture and/or scan the QR code on a screen of user device 104.

In various embodiments, group of databases 102b may be implemented over a blockchain network (such as a PTOYNet blockchain network or a PTOYNet Ethereum™ Blockchain network) and may be present as different databases installed at different locations. The group of databases 102b, which can include Artificial Intelligence Learning Module (AILM) patient database 124 and machine learning database 126, may be configured to store data belonging to different users and data required for functioning of HIE system 102. Different databases can be used in accordance with various embodiments; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. In various embodiments, the data may represent the results of one medical test in a series of multiple medical tests.

In various embodiments, group of databases 102b may operate collectively or individually. Further, group of databases 102b may store data as tables, objects, or other data structures. Further, group of databases 102b may be configured to store data retrieved or processed by HIE system 102. The data may include, but not limited to, a patient medical history, medical charts, medications, prescriptions, immunizations, test results, allergies, insurance provider, or billing information. Further, the data may be time-dependent and piece-wise. Further, the data may represent a subset of data for each patient. In various embodiments, the data may represent results of a medical test in a series of multiple medical tests. Further, the data may be securely stored. In various embodiments, the data may be encrypted.

In various embodiments, information stored in group of databases 102b may be accessed based on users' identities and/or the users' authorities. The users' identities may be verified in one or more ways such as, but not limited to, biometric authentication also known as bioauthentication, password or PIN information, user device registrations, a second-level authentication, or a third-level authentication. In various embodiments, the users' identities may be verified by HIE system 102. Information provided by the users in a real-time may be used, by HIE system 102, to confirm the users' identities. In various embodiments, the users' identities may be verified using a name, a password, one or security questions, or a combination thereof. In various embodiments, a user may be identified using an encryption key and/or a decryption key.

In various embodiments, the data stored in group of databases 102b may be accessed at different levels, for example using a first level subsystem and a second level subsystem. In various embodiments, a user may directly access the first level subsystem. To access data stored in the second level subsystem, the second level subsystem may need to be accessed through the first level subsystem. It should be noted that the communication between the first level subsystem and the second level sub-system may be encrypted. In various embodiments, the second level subsystem may be implemented over a blockchain network (such as a PTOYNet blockchain network). In various embodiments, the PTOYNet blockchain network may be used to implement smart contracts.

In various embodiments, a primary care physician may input data into HIE system 102 using user device 104. The data may be processed by the first level subsystem and the second level subsystem. This may be done successively. The data may be stored on the first level subsystem and/or the second level subsystem of HIE system 102. This may be done successively. The data may include, but not limited to, one or more instructions to a patient to see a physician specialist. Further, the data may be stored in one or more blockchains of the second level subsystem. The patient may be able to access the data relating to the patient's care provided by the primary care physician. This may be done successively. The patient may be able to retrieve the data using user device 104 of the patient. This may be done successively.

In accordance with various embodiments, the patient may communicate with the physician specialist using HIE system 102. It should be noted that the physician specialist may be able to access the data of the patient from the first level subsystem and/or the second level subsystem. Further, the physician specialist may be able to communicate with the patient. It should be noted that some, all (or substantially all) communications between the primary care physician, the physician specialist and the patient may be stored and may be accessible on a blockchain network.

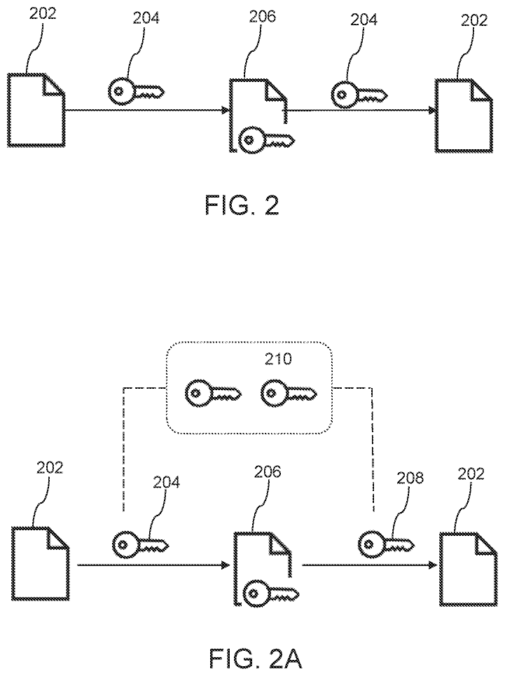

FIG. 2 illustrates a method for symmetric encryption of data, according to various embodiments. Original data 202 may be encrypted using a key 204 to obtain an encrypted data 206. Encrypted data 206 may be decrypted using key 204 to obtain back original data 202. It should be noted that encryption and decryption of the data may be performed using a same key. Further, one or more parties involved in a communication may have the same key to encrypt and decrypt the data.

FIG. 2A illustrates a method for asymmetric encryption of data, according to various embodiments. Original data 202 may be encrypted using key 204 to obtain encrypted data 206. Encrypted data 206 may be decrypted using another key 208 to obtain the original data 202. It should be noted that encryption and decryption of the data may be performed using different keys, such as a key pair 210.

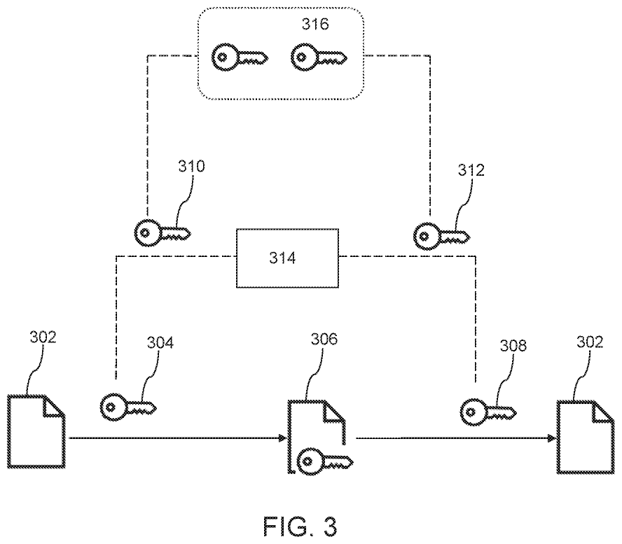

FIG. 3 illustrates a method for hybrid encryption of data, in accordance with various embodiments. Symmetric encryption and asymmetric encryption techniques may be used in tandem. In various embodiments, the symmetric encryption technique may be used to encrypt data 302 using a symmetric key 304 for producing encrypted data 306. Encrypted data 306 may be decrypted using another symmetric key 308 for obtaining data 302, e.g., back data. Further, a public key 310 may be used to encrypt symmetric key 304 and a private key 312 may be used to encrypt symmetric key 308, stored as an encrypted key 314. Public key 310 and private key 312 may form a key pair 316.

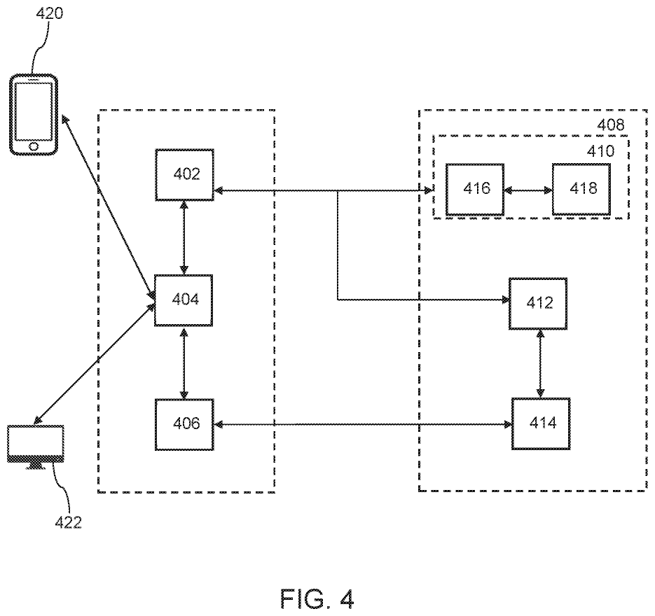

In accordance with various embodiments, referring to FIG. 4 illustrating an example of a system for storing and accessing data in a health care network, the first level subsystem may include a core service component 402 and a Remote Procedure Call (RPC) component 404. In various embodiments, the second level subsystem may include a blockchain node component 406 such as quorum blockchain node component 406. In various embodiments, the first level subsystem may include core service component 402, and the second level subsystem may include RPC component 404 and quorum blockchain node component 406. Further, core service component 402 of the first level subsystem may be present in communication with third-party servers and databases of a hospital computing network 408. Hospital computing network 408 may include an IPFS module 410, an EHR synchronization service 412, and a quorum blockchain node 414. Further, IPFS module 410 may include an IPFS manager 416 and an IPFS node 418. Quorum blockchain node component 406 of the second level subsystem may communicate with blockchain node 414, for example, quorum blockchain node 414 of hospital computing network 408. Patients may access the health care network for storing data through a user device 420, and a representative of a hospital may access the health care network through another user device 422.

In accordance with various embodiments, the representative of the hospital may want to synchronize Electronic Health Record (EHR) data of a patient. The first level subsystem and the second level subsystem may ask the patient for permission to allow a representative of the hospital to store the EHR data of the patient, through IPFS module 410. This may be done successively. Based at least on the permission granted by the patient, a signed transaction may be created to confirm the permission of the hospital to store the EHR data. Further, the signed transaction may activate a smart contract that may add hospital identification information such as a blockchain address to a list of permitted users.

In accordance with various embodiments, the signed transaction may be transmitted from the user device to RPC component 404 of the first level subsystem and/or the second level subsystem. RPC component 404 may communicate the signed transaction to the quorum blockchain node component 406 of the second level subsystem. This may be done successively. Quorum blockchain node component 406 may activate one or more smart contracts. Thereafter, the quorum blockchain node component 406 may revise a state of one or more blockchains. This may be done successively.

In accordance with various embodiments, based at least on the permission granted by the patient, the EHR synchronization service may obtain a list of patients from RPC component 404. The EHR synchronization service may confirm whether the patient has granted permission. Based at least on the permission, the first level subsystem and the second level subsystem may obtain the EHR data and may calculate a hash function for the EHR data. HIE system 102 may match the hash function of the EHR data with a hash function for the patient blockchain on quorum blockchain node component 406 of the second level subsystem. This may be done successively. If the hash function of the EHR data matches with the hash function for the patient blockchain on quorum blockchain node component 406 of the second level subsystem, the EHR data of the patient may remain unchanged.

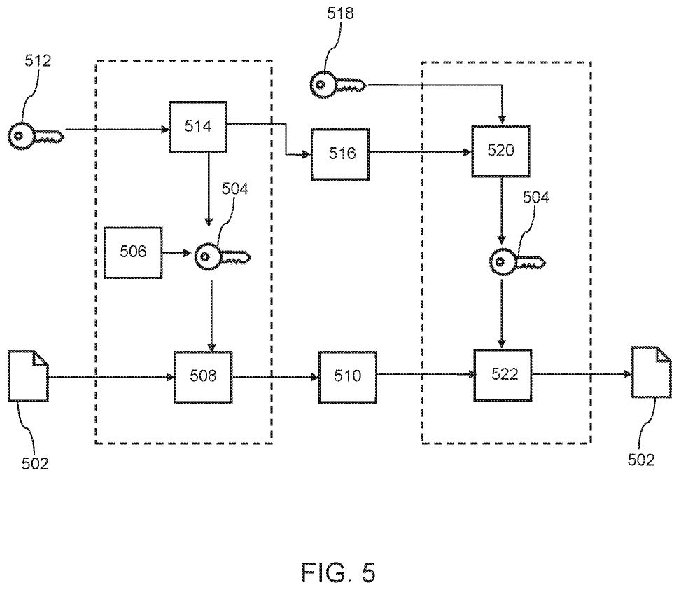

In accordance with various embodiments, referring to FIG. 5 illustrating an example of a system for storing and accessing data in a health care network implemented specifically over a blockchain network (such as a PTOYNet blockchain network or a PTOYNet Ethereum™ Blockchain network), HIE system 102 may execute an application for determining permission from the user for obtaining EHR data 502. In various embodiments, if the user grants the permission, HIE system 102 may obtain EHR data 502 for calculating a hash function for EHR data 502. HIE system 102 may match the hash function of EHR data 502 with a hash function for the user blockchain on the quorum blockchain node of the second level sub-system. In various embodiments, if the two hash matches, there is no change to the user's EHR data 502. In various embodiments, if the two hash functions do not match, HIE system 102 may generate a random string e.g., secret key 504, through a random key generator 506. Secret key 504 may be used for Advanced Encryption Standard (AES) encryption of EHR data 502, in an AES encryptor 508, for generating encrypted EHR data 510.

In accordance with various embodiments, key 504 may then be encrypted by, for example, a Rivest-Shamir-Adleman (RSA) public key 512 of the patient, in an RSA encryptor 514, to generate an encrypted secret key 516. HIE system 102 may also send the encrypted EHR data 510 to core service component 402 for forwarding the data to IPFS manager 416 of hospital computing network 408 for storage. IPFS manager 416 may send an IPFS hash function to core service component 402 for further sending the IPFS hash function to EHR synchronization service 412. EHR synchronization service 412 may further update the patient smart contract with the new IPFS hash function, the encrypted random key, a hash function of the unencrypted file, and file name.

In accordance with various embodiments, a hospital representative, such as a doctor or a hospital administration, may want to view EHR data 502. In such a scenario, the user may first send a signed transaction to a RPC component 404 for granting permission to the hospital representative to view EHR data 502. Once the permission is granted, the signed transaction may be added to the quorum blockchain node 414 and a new smart contract will be created for a blockchain corresponding to the hospital representative. After adding the signed transaction, the hospital representative may be able to view EHR data 502 of the user, on a device.

In various embodiments, to view EHR data 502 on the device, HIE system 102 may collect encrypted EHR data 510 from the user's blockchain and may decrypt encrypted EHR data 510 using patient's RSA private key 518. HIE system 102 may decrypt encrypted secret key 516, in an RSA decryptor 520, using RSA private key of the hospital representative. Encrypted EHR data 510 may be decrypted using RSA public key 512 of the hospital representative, in an AES decryptor 522. This may be done successively. Further, HIE system 102 may load decrypted EHR data 502 to the smart contract previously created for the hospital representative.

After loading decrypted EHR data 502, RPC component 404 may obtain the signed transaction from the patient's user device and transmit the signed transaction to the quorum blockchain node component 406 of the second level subsystem. Quorum blockchain node component 406 may confirm ownership of the signed transaction and may execute the smart contract for the hospital representative to view the user's data.

In various embodiments, the patient may decline permission for the hospital representative to have access to EHR data 502. In such an example scenario, the user, through a user device, may send a signed transaction revoking permission to RPC component 404. RPC component 404 may forward the signed transaction to quorum blockchain node component 406 of the second level subsystem. Quorum blockchain node component 406 may confirm ownership of the signed transaction and may delete the smart contract previously created to allow the hospital representative to have access to patient's EHR data 502.

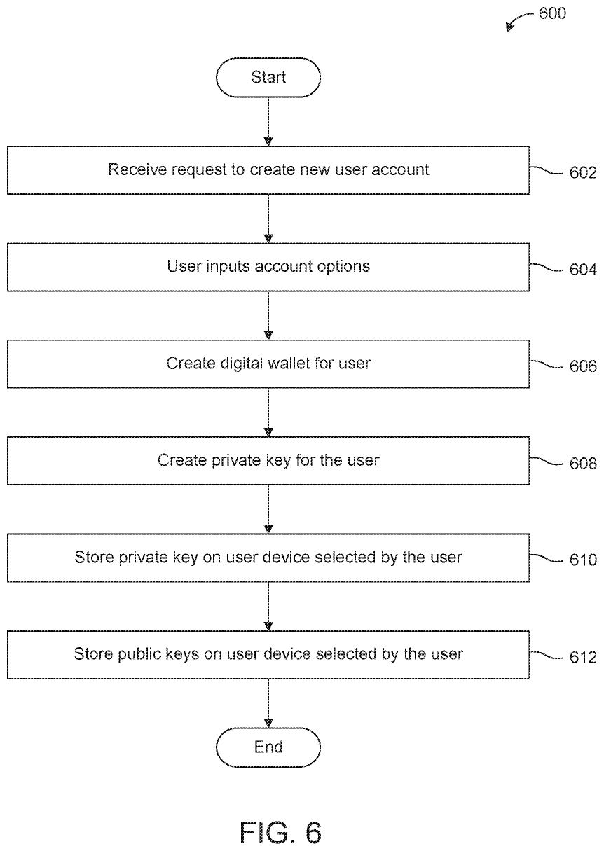

Functioning of patient set up module 118 will now be explained with reference to the example flowchart 600 shown in FIG. 6. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 6, a request may be received from a user to create a new user account, using user device 104, at step 602. Upon receiving the request, the user may input account information (e.g., personal information) into the user account, at step 604. In various embodiments, the personal information may comprise of an e-mail address, address, name, etc. of the new user. Patient set up module 118 may create a digital wallet (e.g., PTOYNet Ethereum wallet) for the user, at step 606. Further, patient set up module 118 may create a private key for the user, at step 608.

Moreover, patient set up module 118 may store the private key on user device 104, at step 610. It should be noted that the private key is stored on user device 104 so that, for example, only the user has access to the private key for modifying or viewing the user's heath record. Further, patient set up module 118 may store public keys corresponding to the user's heath record on user device 104, at step 612. In various embodiments, the user may send the public keys to a third party for proving access to view a selected portion of the user's health record, upon approval by the user.

In various embodiments, machine learning module 120 may collect relevant user's health record from a plurality of users by receiving the public keys from third parties. Machine learning module 120 may allow the third parties to access data of patient (upon request) if the group of patients has provided access. Machine learning module 120 may seek to get the public keys from groups of patients to mine the data stored in the blockchain. In various embodiments, data in machine learning module 120 may be accessed, for example at the front end, and later analyzed by an AI engine.

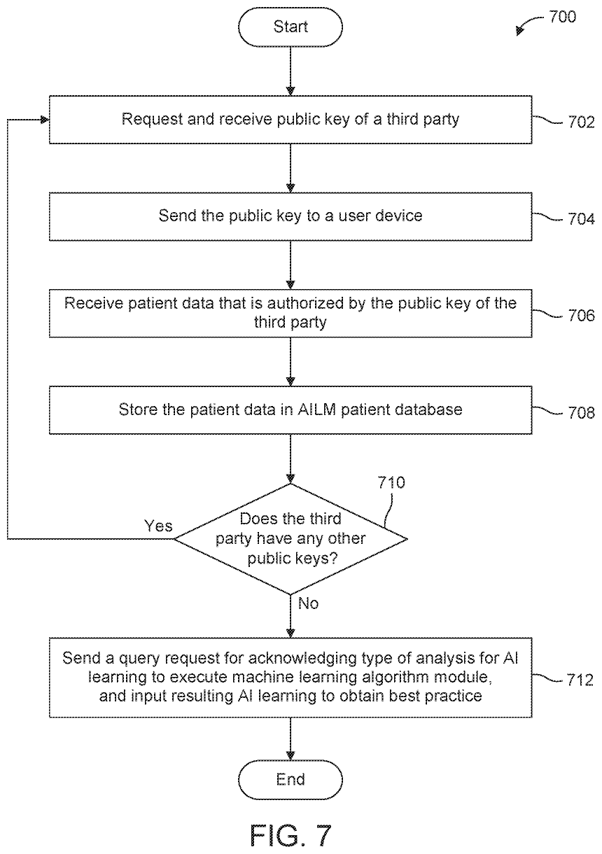

Functioning of machine learning module 120 will now be explained with reference to the example flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Machine learning module 120 may request and receive the public key of the third party, at step 702. In various embodiments, the public keys can be requested for allowing the third parties to view a selected portion of the user's health record. Machine learning module 120 may send the public keys to the user through user's device 104, at step 704. In various embodiments, machine learning module 120 may send the public keys to a device from which the user has created the user account. Machine learning module 120 may receive patient data authorized by the public key of the third parties at step 706. It should be noted that the patient data, while accessed by the user, can be accessed through a different key (e.g., a private key). Further, machine learning module 120 may store the patient data in

AILM patient database 124, at step 708.

Further, machine learning module 120 may determine if the third parties have any other public keys, at step 710. In various embodiments, the third party have any additional key, machine learning module 120 may return control to step 702, for obtaining additional user's patient data and store the additional user's patient data in AILM patient database 124. In various embodiments, when the third parties do not have any other public keys, machine learning module 120 may execute a machine learning algorithm for determining levels of correlation. If the correlation is low, for example, the machine learning module 120 may not be executed. If the correlation is high, for example, the machine learning module 120 may return AI database of best practices to the third parties.

Further, machine learning module 120 may send a query request for acknowledging the type of analysis (e.g., appendicitis) for AI learning to execute machine learning algorithm module 122, and input resulting AI learning to obtain best practices, at step 712. In various embodiments, the machine learning module 120 may send many examples related to a first parameter (e.g., appendicitis) to an AI learning machine for determining high correlation in other parameters or second parameters. It should be understood that any of the parameters may be in a variety of ways such as numerical data or descriptive data that relates to the wellness of the patient in terms of physical and/or mental wellness.

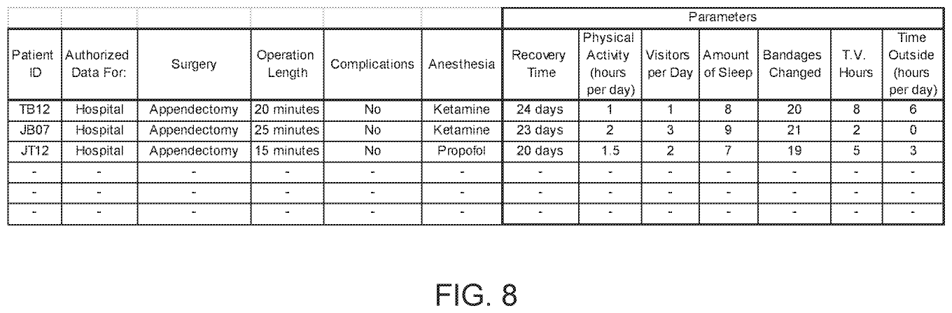

In accordance with various embodiments, FIG. 8 illustrates exemplary data stored in AILM (Artificial Intelligence Learning Module) patient database 124. AILM patient database 124 may comprise patient data received from user device 104 using public key from the 3rd Party, for example, the hospital. The public key may be sent to the user for combining the public key with the user's private key to access the authorized data. The authorized data may be sent to machine learning module 120, which may store the data in AILM patient database 124. AILM patient database 124 may include, for example and not limited to, patient identification and authorized party for which data is intended, surgery type, operation length, complications, anesthesia, recovery time, amount of physical activity per day, visitors per day, amount of sleep per night, number of times while bandages were changed, amount of T.V. watched per day, and time spent outside per day.

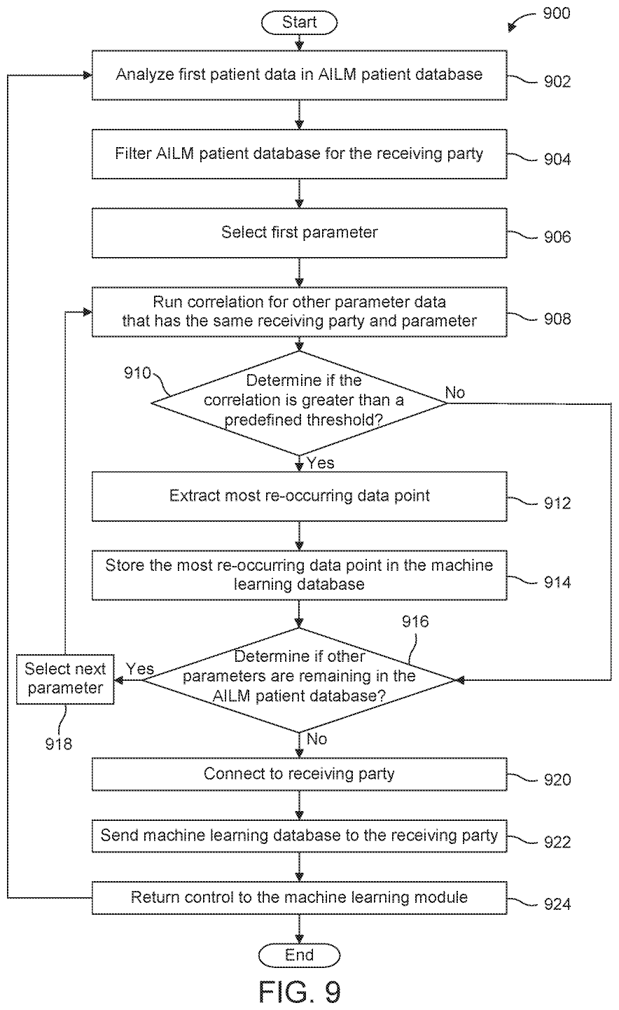

Functioning of machine Learning Algorithm Module (LAM) 122 will now be explained with reference to the example flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 9, patient data stored in AILM patient database 124 may be identified and analyzed, at step 902. In various embodiments, machine LAM 122 may analyze authorized patient data received from user's heath record, through quorum blockchain node component 406. Machine

LAM 122 may filter data stored in AILM patient database 124 for receiving parties including, for example, Hospital, pharmacies, physicians, or insurance companies, at step 904. In various embodiments, machine LAM 122 may allow a selected portion of the patient data in the blockchain to be accessed by the receiving parties. For example, a receiving party e.g., pharmacy may receive patient's current prescription data but may not be able to access patient's medical imaging data.

Machine LAM 122 may select a first parameter, for example, a surgery type, at step 906. In various embodiments, machine LAM 122 may select the first parameter related to the data accessible to the receiving. AILM patient database 124 may be filtered based on the receiving party and the first parameter. Machine LAM 122 may run correlation for other parameter data having same receiving party and parameters e.g., parameter data that is accessible to the receiving party, at step 908. Machine LAM 122 may compare if the correlation is greater than a predefined threshold, at step 910. In various embodiments, the predefined threshold may be set as 95%. Such correlation may be compared for determining relevance of the other parameter data for the receiving party. Any of the parameters may be predetermined as related to the physical and mental wellness of a person.

If the correlation is determined to be present above the predefined threshold, machine LAM 122 may extract the most re-occurring data point, at step 912. For example, if there is a high correlation between patients having lower blood pressure and shorter wait times while waiting to see the physician, the most re-occurring data point may be extracted. Further, machine LAM 122 may store the most re-occurring data point in machine learning database 126, at step 914. In various embodiments, machine LAM 122 may determine if any other parameters, accessible to the receiving party, are present in AILM patient database 124, at step 916. This may occur, for example, if the correlation is determined to be present below the predefined threshold. In various embodiments, if such other parameters may be found to be present, machine LAM 122 may select a next parameter from the other parameters, at step 918. Further, machine LAM 122 may again perform correlations for all of the remaining parameters, at step 908. In various embodiments, if no other parameters are found to be present, machine LAM 122 may connect to the receiving party, at step 920. Further, machine LAM 122 may send machine learning database 126 to the receiving party, at step 922. Machine LAM 122 may return control to machine learning module 120, at step 924.

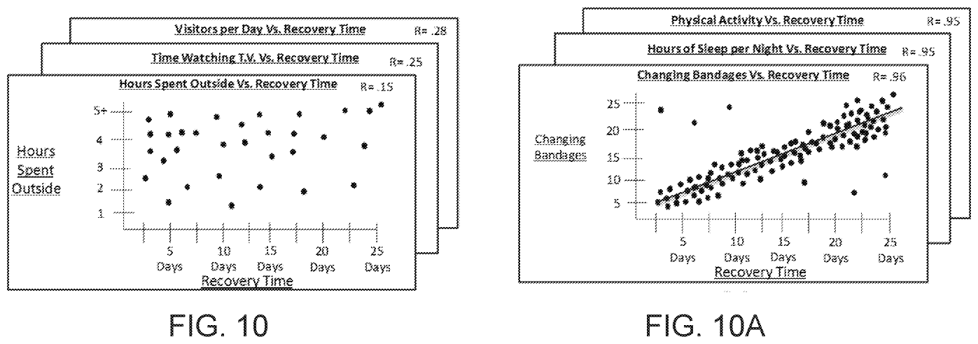

In accordance with various embodiments, FIGS. 10A and 10B illustrate exemplary data stored in the machine learning database 126. FIG. 10A illustrates, for example, correlations performed between recovery and the various other parameters such as hours spent outside, time watching T.V., and visitors per day. For example, referring to example data of FIG. 10A, a parameter that does not correlate with recovery time is hours spent outside, with a correlation of 15%, which would be below a predefined threshold set at 95%. Therefore, no correlation exists in such condition and data points may not be stored in the machine learning database 126.

FIG. 10B illustrates, for example, correlations performed between the recovery time and various parameters such as changing bandages, hours of sleep per night, and physical activity. For example, correlation performed between the recovery time and changing bandages results in a correlation score of 96%, which would be above a predefined threshold set at 95%. Therefore, occurrence of the correlation is identified and a data point corresponding to frequent change of bandages leading to quicker recovery times, may be stored in machine learning database 126. A most re-occurring data point e.g., 15 bandage changes and 15 days of recovery, may be extracted and may be stored in machine learning database 126.

In accordance with various embodiments, HIE system 102 may comprise a health record network for an intermediary enabling sharing of user's medical records with third parties. For enabling sharing, the user may grant specific permissions to the third parties for accessing parts of the user's medical records implemented over a blockchain network. The user may also grant specific permissions to the third parties for accessing the user's medical records. In various embodiments, the third parties may include, for example, any users constituting a value chain, such as hospitals, physicians, nurses, specialists, pharmacies, medical laboratories and testing centers, insurance companies, EMT services etc.

Further, a patient module present in user device 102 may allow the user to receive healthcare products or services related to the user's medical records. In various embodiments, the patient module may be a software application executed on the user device 102. Further, the patient module may also allow the third parties to access personal data of the user.

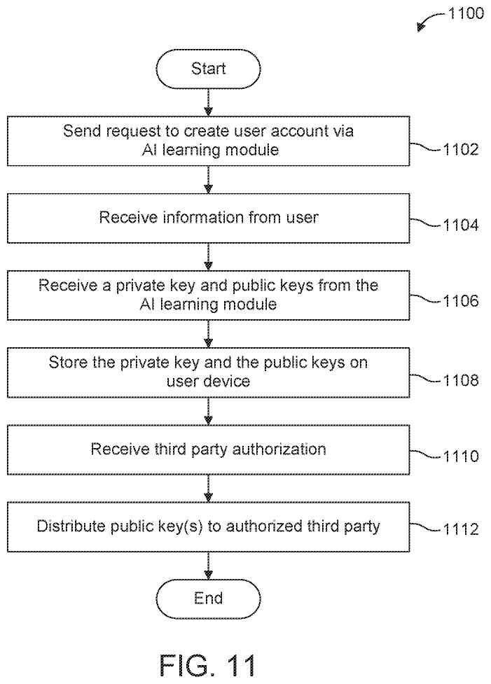

Functioning of the patient module will now be explained with reference to the example flowchart 1100 shown in FIG. 11. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Referring to FIG. 11, the patient module may send a request for creating a user account through an AI (Artificial Intelligence) learning module, at step 1102. Upon sending the request, the patient module may request the user to provide personal information such as an e-mail address, residential or office address, etc., at step 1104. The patient module may receive a private key and public keys from the AI learning module, at step 1106.

The patient module may store the private key and the public keys on user device 104, at step 1108. The private key may be used for accessing the medical records by the user. The public keys may be used for providing access to the third parties to view a selected portion of the user's medical record, for example, upon approval of the user. Further, the patient module may request for a user authorization for selecting a third party that could access the selected portion of the user's medical record, at step 1110. Upon selection, the patient module may distribute the public key(s) to the third parties for accessing the selected portion of user's health record, at step 1112.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for determining best practices for third parties accessing a health care network, comprising:
allowing a patient with a user device to connect to the health care network implemented over a blockchain;
allowing a third party with at least one third party device to connect to the health care network;
receiving, from the third party, a request for accessing data of the patient and parameters as related to the patient, said parameters being a first predetermined parameter related to the patient;
correlating, using an artificial intelligence engine, the first predetermined parameter with a patients' data to identify other of said parameters being a second predetermined parameter relevant for the patient; and
providing the second predetermined parameter to the third party, for being used as a best practice by the third party.

2. The computer-implemented method of claim 1, wherein the third party is an individual belonging to a hospital, insurance company, pharmaceutical company, or a Contract Research Organization, further comprising providing an access right to the patient information based on a type of the patient information and the third party.

3. The computer-implemented method of claim 1, further comprising accessing a digital wallet for the third party to compensate a cost of providing the second predetermined parameter to the third party.

4. The computer-implemented method of claim 1, wherein allowing a third party comprises comparing a public key stored in the third party device with an associated private key stored in a server.

5. The computer-implemented method of claim 1, wherein correlating the first predetermined parameter with patients' data comprises retrieving, with a machine learning module, multiple public keys to access the patients' data from a group of patients in the blockchain.

6. The computer-implemented method of claim 1, wherein correlating the first predetermined parameter with patients' data comprises accessing a list of best practices from an artificial intelligence learning module patient database when a level of correlation is greater than a predefined threshold.

7. The computer-implemented method of claim 1, wherein correlating the first predetermined parameter with patients' data comprises stopping the artificial intelligence engine when a level of correlation is no greater than a predefined threshold.

8. The computer-implemented method of claim 1, wherein correlating the first predetermined parameter with patient's data comprises extracting a highly re-occurring data point.

9. The computer-implemented method of claim 1, wherein correlating the first predetermined parameter with patient's data comprises selecting data with a same receiving party and a same predetermined parameter as the first predetermined parameter from the patient's data.

10. The computer-implemented method of claim 1, wherein correlating the first predetermined parameter with patients' data comprises accessing an artificial intelligence learning module patient database to find the first predetermined parameter and the second predetermined parameter.

11. A system for determining best practices for third parties accessing a health care network, the system comprising:
one or more processors; and
a memory coupled to the one or more processors, the memory including instructions that, when executed by the one or more processors, cause the system to:
allow a patient with a user device to connect to the health care network implemented over a blockchain;
allow a third party with at least one third party device to connect to the health care network;
receive, from the third party, a request for accessing data of the patient and a first predetermined parameter related to the patient;
correlate, analyse, and match, using an Artificial Intelligence (AI) engine, the first predetermined parameter with a patients' data to identify and recommend a second predetermined parameter relevant for the patient; and
send recommendation to the second predetermined parameter to the third party, for being used as a best practice by the third party.

12. The system of claim 11, wherein the third party is an individual belonging to a hospital, insurance company, pharmaceutical company, or a Contract Research Organization, and the one or more processors further execute instructions to provide an access right to the patient information based on a type of the patient information and the third party.

13. The system of claim 11, wherein the one or more processors further execute instructions to access a digital wallet for the third party to compensate a cost of providing the second predetermined parameter to the third party.

14. The system of claim 11, wherein to allow a third party to connect to the health care network the one or more processors execute instructions to compare a public key stored in the third party device with an associated private key stored in a server.

15. The system of claim 11, wherein to correlate the first predetermined parameter with patients' data the one or more processors execute instructions to retrieve, with a machine learning module, multiple public keys to access the patients' data from a group of patients in the blockchain.

16. A non-transitory, computer readable medium storing instructions which, when executed by a computer, cause the computer to perform a method for determining best practices for third parties accessing a health care network, the method comprising:
allowing a patient with a user device to connect to the health care network implemented over a blockchain;
allowing a third party with at least one third party device to connect to the health care network;
receiving, from the third party, a request for accessing data of the patient and a first parameter related to the patient;
correlating, using an artificial intelligence engine, the first parameter with a patients' data to identify a second parameter relevant for the patient;
providing the second parameter to the third party, for being used as a best practice by the third party; and
accessing a list of best practices from an artificial intelligence learning module patient database when a level of correlation is greater than a predefined threshold.

17. The non-transitory, computer readable medium of claim 16 wherein, in the method, correlating the first parameter with patients' data comprises stopping the artificial intelligence engine when a level of correlation is no greater than a predefined threshold.

18. The non-transitory, computer readable medium of claim 16 wherein, in the method, correlating the first parameter with patient's data comprises extracting a highly re-occurring data point.

19. The non-transitory, computer readable medium of claim 16 wherein, in the method, correlating the first parameter with patient's data comprises selecting data with a same receiving party and a same parameter as the first parameter from the patient's data.

20. The non-transitory, computer readable medium of claim 16 wherein, in the method, correlating the first parameter with patients' data comprises accessing an artificial intelligence learning module patient database to find the first parameter and the second parameter.
